(19) United States
(12) Patent Application Publication
(10) Pub. No.: US 2022/0296667 A1
(43) Pub. Date: Sep. 22, 2022

(54) COMPOSITION FOR ALLEVIATING AND TREATING NARCOLEPSY, CONTAINING FERMENTED RICE BRAN POWDER AS ACTIVE INGREDIENT, AND PREPARATION METHOD THEREFOR

(71) Applicant: RAPHAGEN, Seoul (KR)

(72) Inventor: Byoung Hee PARK, Seoul (KR)

(21) Appl. No.: 17/639,069

(22) PCT Filed: Aug. 28, 2019
(86) PCT No.: PCT/KR2019/011002

§ 371 Date: Feb. 28, 2022

(51) Int. Cl. A61K 36/899; A61P 25/26

(52) U.S. Cl. CPC A61K 36/899; A61P 25/26; A61K 2236/19; A61K 2236/51; A61K 2236/53

(57) ABSTRACT

A composition for alleviating and treating narcolepsy, contains a fermented rice bran powder as an active ingredient. The fermented rice bran powder can be obtained by mixing rice bran with a specific strain and fermenting the resulting mixture, and the composition exhibits cranial nerve stimulation and causes no cognitive impairment, thereby exhibiting an effect of waking a sleeping person, and thus can solve psychological and social problems caused by excessive daytime sleepiness and prevent feelings of shame, difficulty in interpersonal relationships, a decline in work performance and a risk of accidents. In addition, the composition is not neurocytotoxic, and thus can achieve only a desired effect without side effects.

### TECHNICAL FIELD

The present invention relates to a composition for alleviating and treating narcolepsy, containing a fermented rice bran powder as an active ingredient, and a preparation method therefor.

### BACKGROUND ART

Sleep disorders include sleep initiation and maintenance disorders (insomnia), narcolepsy, and the like. Narcolepsy is a relatively common nervous system disease that causes serious problems in patients' daily lives due to excessive daytime sleepiness and abnormal REM sleep. Narcolepsy occurs due to abnormalities in the central nervous system, particularly, the hypothalamus, and recently, a decrease in secretion of hypocretin (also called orexin) produced in the posterior hypothalamus has been revealed as one of the causes of narcolepsy. It has been confirmed that 70 to 80% of patients with narcolepsy have very low hypocretin levels in the cerebrospinal fluid. It has also been reported that cerebral glucose metabolism is significantly reduced in the hypothalamus, thalamus, anterior frontal lobe (subcallosal, rectal gyrus), and the like of patients with narcolepsy. Narcolepsy is still difficult to cure, persists chronically, and thus is known to be able to induce a variety of psychological and social complications.

The international classification of sleep disorders revised in 1997 stipulated four major symptoms of narcolepsy as disorders of rapid eye movement (REM) sleep, such as excessive daytime sleepiness, cataplexy, sleep paralysis, and hypnagogic hallucinations (M. N. Rochester, American Sleep Disord. Association. pp 1-52, 1997). Further, many patients with narcolepsy have accompanying impaired night sleep and automatic behavior. Sleepiness caused by narcolepsy usually begins in adolescence, but may also begin in childhood or in the 30s and 40s. Almost all the patients, despite having a good night's sleep, complain of excessive sleepiness in the daytime and often experience sleep attacks in which they suddenly fall asleep during their daily activities. Along with daytime sleepiness, the quality of nighttime sleep also deteriorates.

The second symptom, cataplexy, is a phenomenon in which all or part of the body suddenly loses strength when there are emotional changes such as laughing, crying, anger, joy, or liking, or standing with a sudden loss of strength, and is a symptom in which the knees suddenly loosen and the muscles of the chin or face are weakened. The duration is short, within seconds to minutes, and patients quickly recover completely. Consciousness is maintained and all situations can be remembered.

Sleep paralysis known as ‘ghost press’ refers to a state of immobility for several seconds to several minutes when falling asleep or waking from sleep. About 25% of patients with narcolepsy have accompanying sleep paralysis. In this case, the patient is awake, but he or she cannot move his or her limbs, and thus feels anxiety and fear and also sees horrifying hallucinations. Sleep paralysis either ends spontaneously or disappears with a slight stimulus. Normal people also experience sleep paralysis once or more, and often have particularly irregular sleep habits. Sleep paralysis may occur alone when there is a family history.

A hypnagogic hallucination is a phenomenon in which a dream turns into reality, an illusion is seen, or an auditory hallucination is heard when a person is trying to fall asleep after awakening, or in the intermediate stage of trying to wake up. Hypnagogic hallucinations are mostly scary or unpleasant. Although in a state of hallucinations, consciousness is maintained and all surroundings can be recognized. Hypnagogic hallucinations can also be experienced by normal individuals, and are experienced by 30% of patients with narcolepsy.

Narcolepsy treatment is divided into behavioral treatment and drug treatment, and behavioral treatment uses methods such as maintaining a regular sleep-wake cycle and ensuring sleep hygiene. Drug treatment is divided into three aspects: the first is the treatment of hypersomnia using central nervous system stimulants, the second is the treatment of REM sleep-related symptoms such as cataplexy, sleep paralysis, and hypnagogic hallucinations, and the last is regulation for poor night sleep.

As drugs for treating hypersomnia, amphetamines, methylphenidate, pemoline, and the like are typical symphathomimetic drugs, but their use is restricted due to their dependence and side effects.

Caffeine and modafinil are typical central nervous system stimulants rather than symphathomimetic drugs. Caffeine is a natural alkaloid, and is most widely used for its awakening effect. Caffeine enhances awakening by suppressing sleep-inducing adenosine receptors. One cup of instant coffee contains 40 to 105 mg of caffeine, and one cup of decaffeinated coffee contains 1 to 4 mg of caffeine. Since caffeine has a half-life of 3 to 12 hours, ingesting coffee late in the afternoon may induce insomnia.

Modafinil is a typical therapeutic agent for narcolepsy and is easier to take than existing drugs because modafinil has a half-life of 15 hours or more and is taken once a day. More than 80 to 90% of modafinil is metabolized by the liver, plasma concentration is maximized after 2 to 4 hours of taking modafinil, and patients with poor livers are recommended to use about ½ of the usual dose.

Gamma hydroxybutyrate (hereinafter referred to as GHB) is used as a therapeutic agent for nighttime sleep, and is known to be involved in consolidate sleep by increasing both REM sleep and slow-wave sleep as an endogenous material involved in sleep induction. It has been reported that taking this drug ameliorates symptoms such as daytime sleepiness, cataplexy, ghost press, hypnagogic hallucinations, and sleep difficulties, and gamma hydroxybutyrate is a therapeutic agent for cataplexy, which is currently the only one approved by the FDA (USA) because its effect for cataplexy is also outstanding. However, since GHB has a short half-life, it should be taken immediately before falling asleep and a secondary dose during sleep is required, examples of side effects include gastrointestinal disorders, weight loss, drowsiness, and the like, and care should be taken because the use of an amount exceeding a proper dose can lead to convulsions or death. Therefore, there is a need for developing a drug for treating narcolepsy with less side effects.

In particular, narcolepsy often occurs in adolescence, and thus may become a psychological and social problem because a patient has difficulties in his or her work or school life due to isolation from society, long absences, feelings of shame for his or her sleepiness and cataplexy, difficulty in interpersonal relationships, a decline in work performance, a risk of accidents at home or in workplaces, a prejudice of being lazy at work or school, and the like caused by excessive daytime sleepiness, which is a main symptom of narcolepsy, so that there is a need for developing a therapeutic agent capable of ameliorating excessive sleepiness symptoms accompanying narcolepsy.

Meanwhile, the present inventors found that a rice bran powder obtained by mixing rice bran with a specific strain and fermenting the resulting mixture alleviates or treats excessive drowsiness symptoms, thereby completing the present invention.

### DISCLOSURE

### Technical Problem

An object of the present invention is to provide a composition for alleviating and treating narcolepsy, containing a fermented rice bran powder as an active ingredient, and a preparation method therefor.

### Technical Solution

An exemplary embodiment of the present invention for achieving the object of the present invention as described above provides a method for preparing a fermented rice bran powder, the method including: a) obtaining a fermented rice bran broth by mixing rice bran with one or more strains and fermenting the resulting mixture; b) purifying the fermented rice bran broth, and then vacuum-concentrating the purified fermented rice bran broth; c) obtaining only solid matter by slurrying or recrystallizing the vacuum-concentrated fermented rice bran broth; and d) obtaining a fermented rice bran powder by drying the solid matter and crushing the dried solid matter.

Hereinafter, the present invention will be described in detail.

First, a fermented rice bran broth is obtained by mixing rice bran with one or more strains (Step a).

Rice bran refers to a fine rice husk that is separated by the process of extracting rice husks from rice and then milling brown rice into white rice. Specifically, rice bran collectively refers to the pericarp, seed coat, and aleurone layer produced when brown rice is milled to make polished white rice, the analysis of a standard chemical composition of rice bran shows 13.5% water, 13.2% protein, 18.3% fat, 38.3% glycose, 7.8% fiber, and 8.9% ash, 2.5 mg of vitamin B1 is contained in 100 g of rice bran, and a large amount of vitamin E is also included.

Meanwhile, in order to prepare a fermented rice bran powder according to an exemplary embodiment of the present invention, first, a process of mixing rice bran with a specific strain and fermenting the resulting mixture is performed, and in this case, the strain according to an exemplary embodiment of the present invention that is usable may be one or more selected from the group consisting of Lactobacillus buchneri, Lactobacillus paracasei subsp. tolerans, Lactobacillus harbinensis, Saccharomycopsis fibuligera and Pichia kudriavzevii. Specifically, it is possible to use a mixed strain in which the above five types of strains are mixed, and when a fermented liquid is obtained using the five types of mixed strains as described above, a fermented rice bran powder to be obtained afterwards allows the present invention to obtain the desired effect of alleviating and treating narcolepsy.

As an example, a fermented rice bran broth may be prepared by injecting a mixed strain in which the above five strains are mixed into 100 g of dried rice bran and 1 L of water, followed by mixing, and then fermenting the resulting mixture under a temperature condition of 40° C., specifically a temperature condition of 37° C. for 46 to 50 hours, specifically 48 hours.

Next, the fermented rice bran broth obtained by the process is purified and vacuum-concentrated (Step b).

Meanwhile, impurities may be primarily removed by a filter in Step b. After impurities are primarily removed by the filter, an additional purification process may be performed. As an example, the purification process may be performed on the fermented rice bran broth using a mixture of water and an alcohol in chromatography including one or more adsorptive resins selected from an epoxy resin and a copolymer of styrene and divinyl benzene.

Meanwhile, in the mixture of water and an alcohol, the amount of water may be 0.5- to 20-fold larger than the amount of the alcohol. Meanwhile, according to an exemplary embodiment of the present invention, the alcohol may be one or more selected from ethanol and methanol.

More specifically, 1000 ml of a fermented rice bran product initially filtered by a filter is first fractionated, may be second fractionated with 500 ml of 10% ethanol, may be third fractionated with 500 ml of 50% ethanol, may be fourth fractionated with 500 ml of 50% ethanol, may be fifth fractionated with 500 ml of 80% ethanol, and may be sixth fractionated with 500 ml of 100% ethanol, but is not particularly limited. Similarly, methanol can also be used in the same manner as the above ethanol.

Meanwhile, PB-600 manufactured by Sicomin Epoxy Systems may be used as the epoxy resin, and HP-20 manufactured by Mitsubishi Chemical Advanced Materials may be used as the copolymer of styrene and divinyl benzene.

Meanwhile, the fermented rice bran broth subjected to the purification process may be vacuum-concentrated under a temperature condition of 65° C. In this case, the vacuum-concentration method is not particularly limited, and may be performed by a method generally used in the art.

Next, only solid matter is obtained by slurrying or recrystallizing the vacuum-concentrated fermented rice bran broth (Step c).

Specifically, a slurry may be formed by adding ethanol or methanol to the vacuum-concentrated fermented rice bran broth, and then filtering again to remove the liquid, leaving only the solid matter. Alternatively, crystals may be precipitated by adding water to the vacuum-concentrated fermented rice bran broth to dissolve the liquid, adding ethanol or methanol thereto, and then filtering again to remove the liquid, leaving only the solid matter.

Finally, a fermented rice bran powder is obtained by drying the solid matter and crushing the dried solid matter (Step d).

Specifically, the solid matter obtained through Step c may be dried by hot air at 60 to 70° C. for 24 hours, or vacuum dried at 40 to 45° C. for 24 hours to completely dry the solid matter. A fermented rice bran powder is obtained by pulverizing the dried solid matter through a crushing process. In this case, the drying process or crushing process method is not particularly limited, and may be performed by a method generally used in the art.

Another exemplary embodiment of the present invention provides a composition for alleviating and treating narcolepsy, containing a fermented rice bran powder prepared by the method as an active ingredient.

Meanwhile, the fermented rice bran powder may be included in the composition for alleviating and treating narcolepsy in an amount of 1 to 50 wt % based on the total weight of the composition, and when the fermented rice powder is included within the above wt %/o range, an effect of effectively alleviating and treating narcolepsy can be expected.

The composition containing the fermented rice bran powder according to an exemplary embodiment of the present invention described above as an active ingredient exhibits cranial nerve stimulation and causes no cognitive impairment like pentylenetetrazole (PTZ) that is a narcolepsy-related drug, and thus can solve psychological and social problems caused by excessive daytime sleepiness and prevent feelings of shame, difficulty in interpersonal relationships, a decline in work performance and a risk of accidents. In addition, the composition containing the fermented rice bran powder according to the present invention as an active ingredient is not neurocytotoxic, and thus can achieve only a desired effect of alleviating and treating narcolepsy such as daytime sleepiness without side effects.

### Advantageous Effects

Provided in the present invention is a composition containing, as an active ingredient, a fermented rice bran powder obtained by mixing rice bran with a specific strain and fermenting the resulting mixture, and the composition exhibits cranial nerve stimulation and causes no cognitive impairment, thereby exhibiting an effect of waking a sleeping person, and thus can solve psychological and social problems caused by excessive daytime sleepiness and prevent feelings of shame, difficulty in interpersonal relationships, a decline in work performance and a risk of accidents. In addition, the composition according to the present invention is not neurocytotoxic, and thus can achieve only a desired effect without side effects.

### DESCRIPTION OF DRAWINGS

FIGS. 1 and 2 are graphs showing the measured locomotor activity of zebrafish treated with the fermented rice bran powder obtained according to an exemplary embodiment of the present invention.

FIGS. 3 and 5 are graphs showing the measured dark/light transition of zebrafish treated with the fermented rice bran powder obtained according to an exemplary embodiment of the present invention.

FIGS. 6 and 10 are graphs showing the measured color preference of zebrafish treated with the fermented rice bran powder obtained according to an exemplary embodiment of the present invention.

### MODES OF THE INVENTION

A method for preparing a fermented rice bran powder, the method including: a) obtaining a fermented rice bran broth by mixing rice bran with one or more strains and fermenting the resulting mixture;

b) purifying the fermented rice bran broth, and then vacuum-concentrating the purified fermented rice bran broth;

c) obtaining solid matter by slurrying or recrystallizing the vacuum-concentrated fermented rice bran broth; and

d) obtaining a fermented rice bran powder by drying the solid matter and crushing the dried solid matter.

### DETAILED DESCRIPTION OF PREFERRED EMBODIMENTS

Since the present invention may be modified into various forms and include various exemplary embodiments, specific exemplary embodiments will be illustrated and described in detail below. However, the description is not intended to limit the present invention to the specific disclosed forms, and it is to be understood that all the changes, equivalents and substitutions included in the spirit and scope of the present invention are included in the present invention.

Hereinafter, the method for preparing a fermented rice bran powder according to specific exemplary embodiments of the present invention and a composition for alleviating and treating narcolepsy, containing the fermented rice bran powder prepared by the method as an active ingredient will be described in more detail.

### Example 1

A mixed strain in which Lactobacillus buchneri, Lactobacillus paracasei subsp. tolerans, Lactobacillus harbinensis, Saccharomycopsis fibuligera and Pichia kudriavzevii were mixed was inoculated into 100 g of rice bran and 1 L of water, and the inoculated rice bran was fermented at a temperature of 37° C. for 48 hours. Impurities were removed from the obtained fermented rice bran broth by filtration using diatomaceous earth and a centrifuge.

A chromatography column containing 300 g of PB-600 was filled with this fermented product, and the ratio of ethanol and water was sequentially developed at 10%, 30%, 50%, 80%, and 100% for purification. This purified product was vacuum-concentrated at a temperature of 65° C.

Only solid matter was obtained by adding 500 mL of ethanol to 50 g of the obtained fermented rice bran broth to form a slurry, and then filtering the slurry through a Nutsche filter.

A fermented rice bran powder was obtained by crushing a product obtained by drying the obtained solid matter with hot air at a temperature of 70° C. for 2 hours with a crusher for pulverization.

### Example 2

A mixed strain in which Lactobacillus buchneri, Lactobacillus paracasei subsp. tolerans, Lactobacillus harbinensis, Saccharomycopsis fibuligera and Pichia kudriavzevii were mixed was inoculated into 100 g of rice bran and 1 L of water, and the inoculated rice bran was fermented at a temperature of 37° C. for 48 hours. Impurities were removed from the obtained fermented rice bran broth by filtration using diatomaceous earth and a centrifuge.

A chromatography column containing 300 g of PB-600 was filled with this fermented product, and the ratio of methanol and water was sequentially developed at 10%, 30%, 50%, 80%, and 100% for purification. This purified product was vacuum-concentrated at a temperature of 65° C.

Only solid matter was obtained by adding 500 mL of ethanol to 50 g of the obtained fermented rice bran broth to form a slurry, and then filtering the slurry through a Nutsche filter.

A fermented rice bran powder was obtained by crushing a product obtained by drying the obtained solid matter with hot air at a temperature of 70° C. for 2 hours with a crusher for pulverization.

### Example 3

A mixed strain in which Lactobacillus buchneri, Lactobacillus paracasei subsp. tolerans, Lactobacillus harbinensis, Saccharomycopsis fibuligera and Pichia kudriavzevii were mixed was inoculated into 100 g of rice bran and 1 L of water, and the inoculated rice bran was fermented at a temperature of 37° C. for 48 hours. Impurities were removed from the obtained fermented rice bran broth by filtration using diatomaceous earth and a centrifuge.

A chromatography column containing 300 g of PB-600 was filled with this fermented product, and the ratio of ethanol and water was sequentially developed at 10%, 30%, 50%, 80%, and 100% for purification. This purified product was vacuum-concentrated at a temperature of 65° C.

Only solid matter was obtained by adding 100 ml of water and 500 mL of ethanol to 50 g of the obtained fermented rice bran broth, recrystallizing the resulting mixture, and then filtering the resulting product through a Nutsche filter.

A fermented rice bran powder was obtained by crushing a product obtained by drying the obtained solid matter with hot air at a temperature of 70° C. for 2 hours with a crusher for pulverization.

### Example 4

A mixed strain in which Lactobacillus buchneri, Lactobacillus paracasei subsp. tolerans, Lactobacillus harbinensis, Saccharomycopsis fibuligera and Pichia kudriavzevii were mixed was inoculated into 100 g of rice bran and 1 L of water, and the inoculated rice bran was fermented at a temperature of 37° C. for 48 hours. Impurities were removed from the obtained fermented rice bran broth by filtration using diatomaceous earth and a centrifuge.

A chromatography column containing 300 g of PB-600 was filled with this fermented product, and the ratio of methanol and water was sequentially developed at 10%, 30%, 50%, 80%, and 100% for purification. This purified product was vacuum-concentrated at a temperature of 65° C.

Only solid matter was obtained by adding 100 ml of water and 500 mL of ethanol to 50 g of the obtained fermented rice bran broth, recrystallizing the resulting mixture, and then filtering the resulting product through a Nutsche filter.

A fermented rice bran powder was obtained by crushing a product obtained by drying the obtained solid matter with hot air at a temperature of 70° C. for 2 hours with a crusher for pulverization.

### Comparative Example 1

Pentylenetetrazol (PTZ), which is known as a material that induces artificial seizures in zebrafish, was prepared.

### Experiment 1: Measurement of Locomotor Activity by Fermented Rice Bran Powder of Present Invention

Zebrafish are vertebrates and very similar to humans in terms of genetic composition, and thus have most of the genes that humans have. Thus, in the present invention, the locomotor activity of zebrafish treated with the fermented rice bran powder obtained according to Example 1 was measured. Specifically, 5 dpf zebrafish were prepared in 96 wells and treated with the fermented rice bran powder according to Example 1, and then DanioVision recording & tracking was performed for 30 to 60 minutes, and the obtained data was profiled.

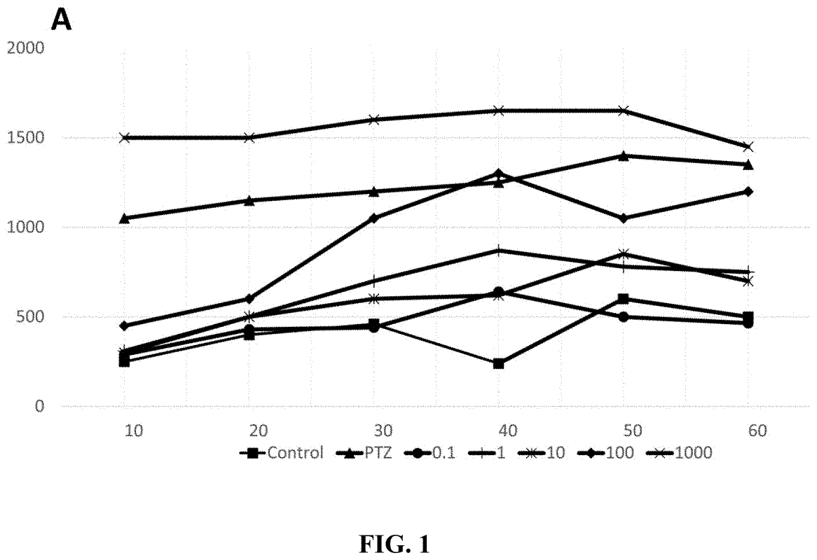

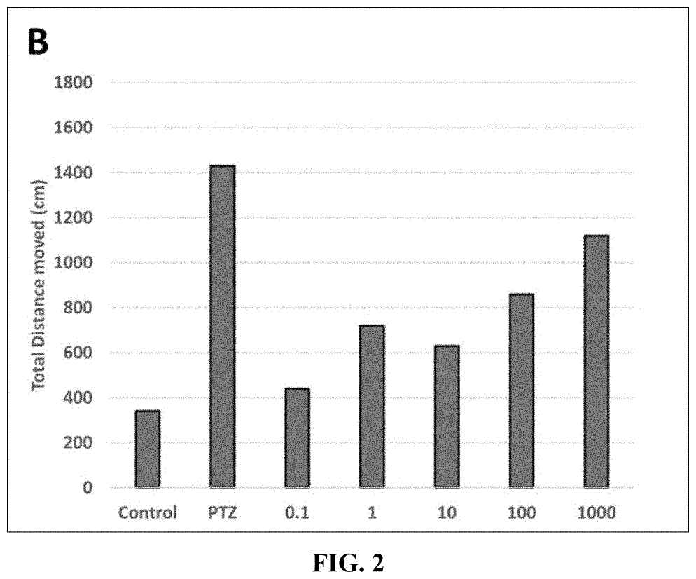

The results are shown in FIGS. 1 and 2, and it could be confirmed that the locomotor activity increased according to the concentration of the fermented rice bran powder according to Example 1. Particularly, it could be confirmed that when the concentration was 1 mg/mL, the fermented rice bran powder was better than PTZ which is a control material (see FIGS. 1 and 2).

### Experiment 2: Measurement of Dark/Light Transition by Fermented Rice Bran Powder of Present Invention

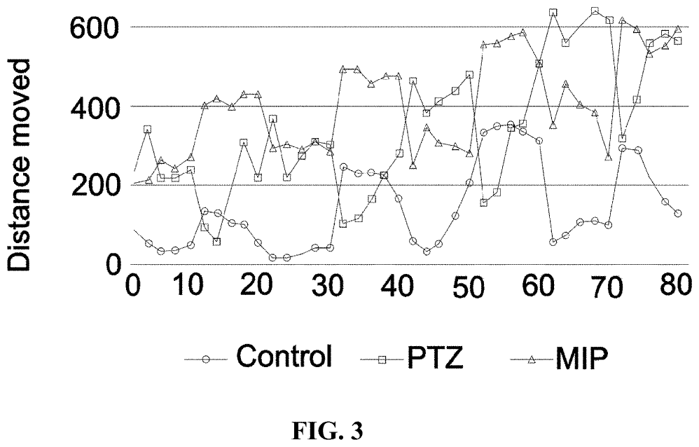

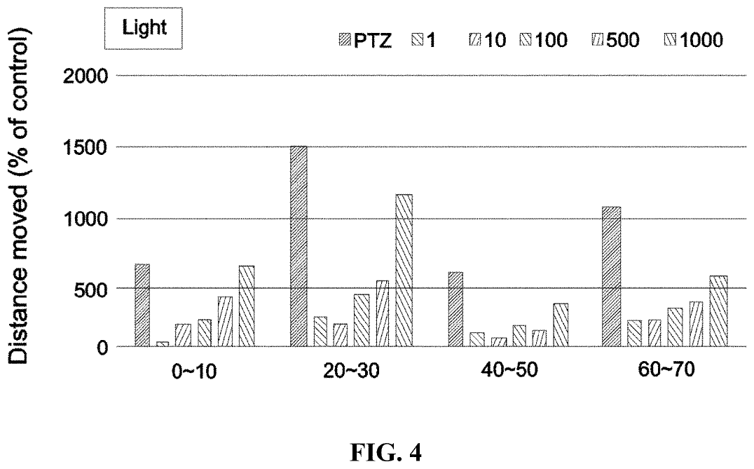

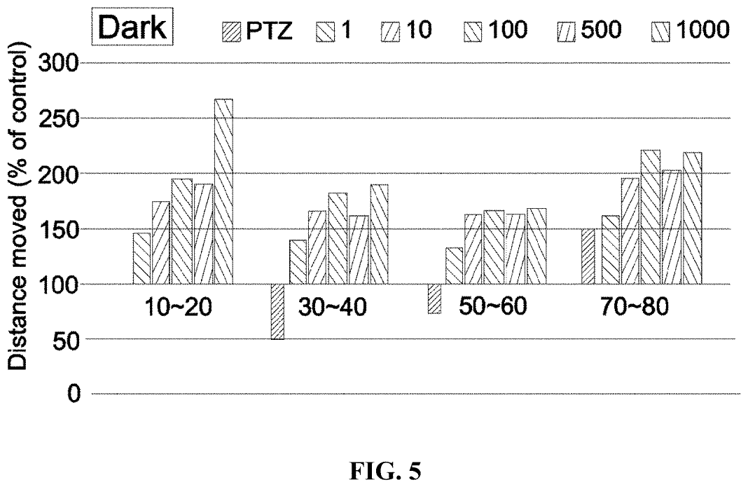

The dark/light transition of zebrafish treated with the fermented rice bran powder obtained according to Example 1 was measured. Specifically, dark and light conditions were given at 10-minute intervals, and graphs for the distance moved at 2-minute intervals were measured (see FIGS. 3, 4, and 5). Referring to FIGS. 3, 4, and 5, it could be confirmed that the control material PTZ moved regardless of the light/dark division, whereas zebrafish treated with the fermented rice bran powder had no impaired cognitive ability, and thus exhibited a normal behavior pattern to dark/light.

### Experiment 3: Measurement of Color Preference by Fermented Rice Bran Powder of Present Invention

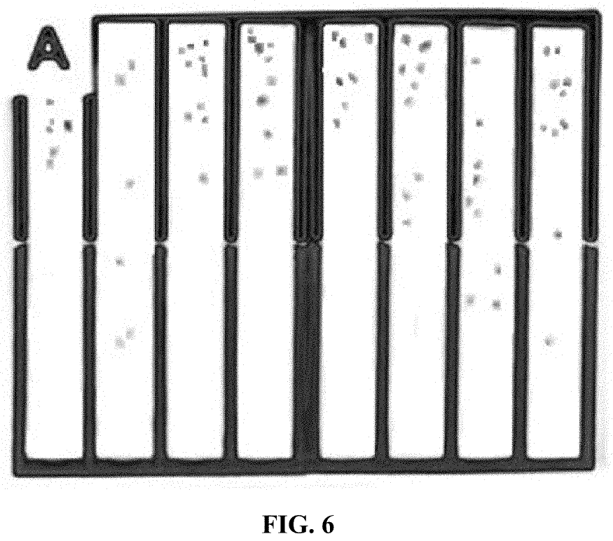

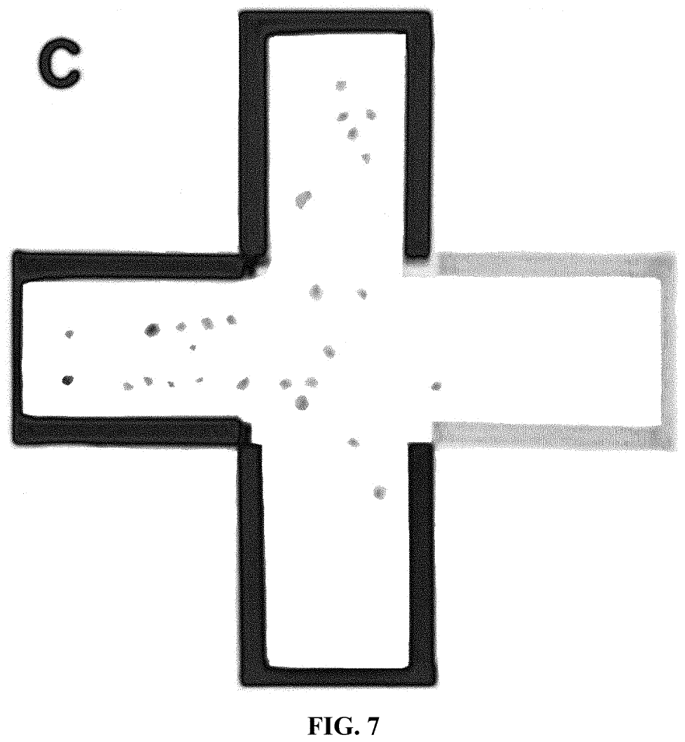

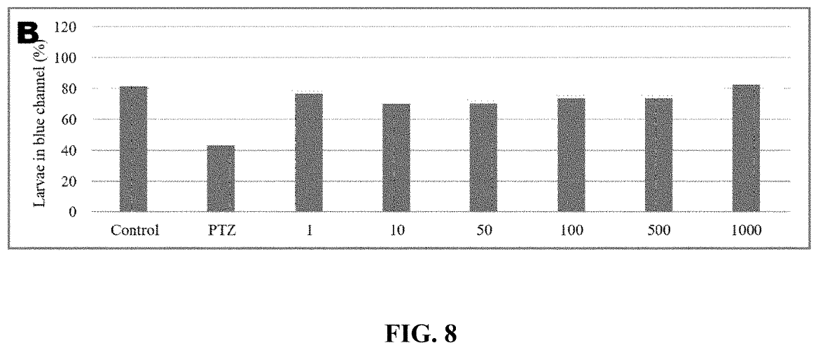

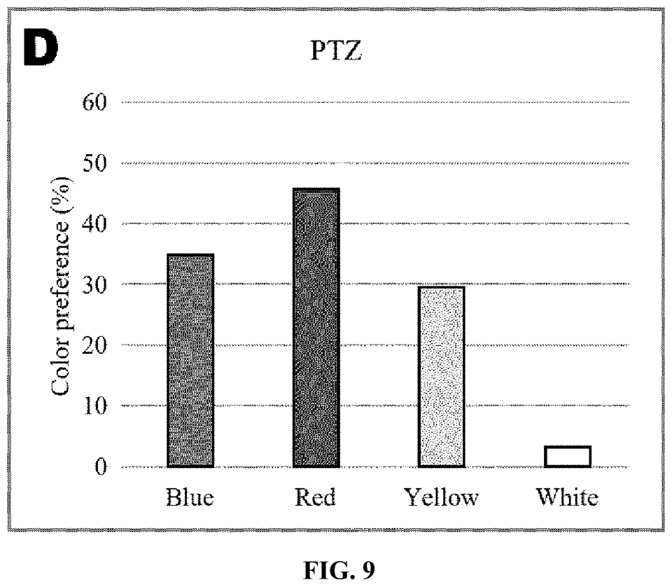

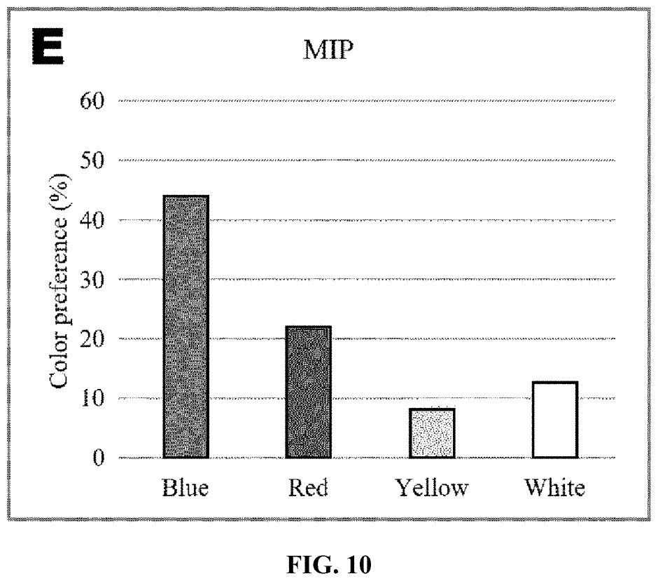

The color preference of zebrafish treated with the fermented rice bran powder obtained according to Example 1 was measured. It is known that in the case of normal zebrafish, the blue preference is excellent, whereas in the case of zebrafish whose brain is damaged by PTZ or the like, color cannot be normally recognized. Specifically, it could be confirmed that when zebrafish was treated with the fermented rice bran powder according to an exemplary embodiment of the present invention, the blue preference is excellent (see FIGS. 6, 7, 8, 9 and 10).

Although examples of the present invention have been described above, a person with ordinary skill in the art to which the present invention pertains will be able to modify the examples of the present invention without departing from the principle or spirit of the present invention. Therefore, it is to be understood that the scope of the present invention is not limited to the described examples, various modifications and alterations can be made without departing from the spirit and scope of the present invention, and such modifications or alterations fall within the present invention.

### INDUSTRIAL APPLICABILITY

Provided in the present invention is a composition containing, as an active ingredient, a fermented rice bran powder obtained by mixing rice bran with a specific strain and fermenting the resulting mixture, and the composition exhibits cranial nerve stimulation and causes no cognitive impairment, thereby exhibiting an effect of waking a sleeping person, and thus can solve psychological and social problems caused by excessive daytime sleepiness and prevent feelings of shame, difficulty in interpersonal relationships, a decline in work performance and a risk of accidents. In addition, the composition according to the present invention is not neurocytotoxic, and thus can achieve only a desired effect without side effects.

1. A method for preparing a fermented rice bran powder, the method comprising: a) obtaining a fermented rice bran broth by mixing rice bran with one or more strains and fermenting the resulting mixture; b) purifying the fermented rice bran broth, and then vacuum-concentrating the purified fermented rice bran broth; c) obtaining only solid matter by slurrying or recrystallizing the vacuum-concentrated fermented rice bran broth; and d) obtaining a fermented rice bran powder by drying the solid matter and crushing the dried solid matter.

2. The method of claim 1, wherein the strain in Step a is one or more selected from the group consisting of Lactobacillus buchneri, Lactobacillus paracasei subsp. tolerans, Lactobacillus harbinensis, Saccharomycopsis fibuligera and Pichia kudriavzevii.

3. The method of claim 1, wherein the fermentation in Step a is performed under a temperature condition of 35 to 40° C. for 46 to 50 hours.

4. The method of claim 1, wherein the purification process in Step b is performed on the fermented rice bran broth using a mixture of water and an alcohol in chromatography comprising one or more adsorptive resins selected from an epoxy resin and a copolymer of styrene and divinyl benzene.

5. The method of claim 4, wherein in the mixture of water and the alcohol, the amount of water is 0.5- to 20-fold larger than the amount of the alcohol.

6. A composition for alleviating and treating narcolepsy, comprising a fermented rice bran powder prepared by the method of claim 1 as an active ingredient.
